(19) United States
(12) Patent Application Publication
(10) Pub. No.: US 2006/0276419 A1
(43) Pub. Date: Dec. 7, 2006

(54) Combined use of ribavirin and interferon beta in demyelinating diseases

(75) Inventor: Giampiero De Luca, Conches (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding n.v., Curacao (NL)

(21) Appl. No.: 10/546,843

(22) PCT Filed: Feb. 24, 2004
(86) PCT No.: PCT/EP04/50202

§ 371 Date: Jun. 30, 2006

(30) Foreign Application Priority Data
Feb. 25, 2003 (EP) 03100459.1

(51) Int. Cl. A61K 31/7056; A61K 38/21

(52) U.S. Cl. 514/43; 424/85.6

(57) ABSTRACT

The present invention is in the field of neurological disorders. It relates to the use of a compound of formula (I) 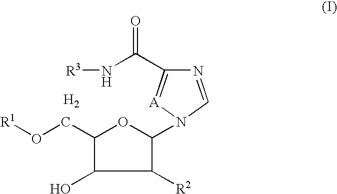 in combination with an interferon (IFN) for the manufacture of a medicament for treatment and/or prevention of a demyelinating disease. In particular, it relates to the use of a combination of Ribavirin and IFN-beta for treatment and/or prevention of a demyelinating disease, such as multiple sclerosis.

### FIELD OF THE INVENTION

The present invention is in the field of neurological disorders. It relates to the use of a compound formula (I) 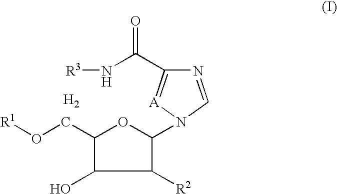
in combination with an interferon (IFN) for the manufacture of a medicament for treatment and/or prevention of a demyelinating disease. In particular, it relates to the use of a combination of Ribavirin and IFN-beta for treatment and/or prevention of a demyelinating disease, such as multiple sclerosis (MS).

### BACKGROUND OF THE INVENTION

Demyelinating diseases are disorders concerning the myelin sheaths of the nervous system. Myelin sheaths, which cover many nerve fibers, are composed of lipoprotein layers formed in early life. Myelin is formed by the oligodendroglia in the CNS and promotes transmission of a neural impulse along an axon.

Many congenital metabolic disorders (e.g. phenylketonuria other aminoacidurias; Tay-Sachs, Niemann-Pick, and Gaucher's diseases; Hurier's syndrome; Krabbe's disease and other leukodystrophies) affect the developing myelin sheath, mainly in the CNS. Unless the biochemical defect can be corrected or compensated for, permanent, often widespread, neurological deficits result.

Demyelination in later life is a feature of many neurological disorders; it can result from damage to nerves or myelin due to local injury, ischemia, toxic agents, or metabolic disorders. Extensive myelin loss is usually followed by axonal degeneration and often by cell body degeneration, both of which may be irreversible. However, remyelination occurs in many instances, and repair, regeneration, and complete recovery of neural function can be rapid. Recovery often occurs after the segmental demyelination that characterizes many peripheral neuropathies; this process may account for the exacerbations and remissions of multiple sclerosis (MS). Central demyelination (i.e. of the spinal cord, brain, or optic nerves) is the predominant finding and in the primary demyelinating diseases, whose etiology is unknown. The most well known demyelinating disease is MS (see below).

Further demyelinating diseases comprise:

Acute disseminated encephalomyelitis, which is characterized by perivascular CNS demyelination, and which can occur spontaneously but usually follows a viral infection or viral vaccination;

Acute inflammatory peripheral neuropathies that follow a viral vaccination or the Guillain-Barré syndrome, they affect only peripheral structures;

Adrenoleukodystrophy and adrenomyeloneuropathy, which are rare X-linked recessive metabolic disorders characterized by adrenal gland dysfunction and widespread demyelination of the nervous system;

Leber's hereditary optic atrophy and related mitochondrial disorders, which are characterized primarily by bilateral loss of central vision, and which can resemble the is optic neuritis in MS; and

HTLV-associated myelopathy, a slowly progressive spinal cord disease associated with infection by the human T-cell lymphotrophic virus, that is characterized by spastic weakness of both legs.

Multiple sclerosis (MS) is a slowly progressive CNS disease characterized by disseminated patches of demyelination in the brain and spinal cord, resulting in multiple and varied neurological symptoms and signs, usually with remissions and exacerbation (see The Merck Manual Home Edition, www.merck.com).

The cause is unknown but an immunological abnormality is suspected, with few clues presently indicating a specific mechanism. Postulated causes include infection by a slow or latent virus, and myelinolysis by enzymes. IgG is usually elevated in the CSF, and elevated titers have been associated with a variety of viruses, including measles. The significance of these findings and of reported associations with HLA allotypes and altered number of T cells is unclear, and the evidence somewhat conflicting. An increased family incidence suggests genetic susceptibilty; women are somewhat more often affected than men. Environmental factors seem to be present. Although age at onset generally is from 20 to 40 years, MS has been linked to the geographic area where a patient's first 15 years are spent. Relocation after age 15 does not alter the risk.

Plaques or islands of demyelination with destruction of oligodendroglia and perivascular inflammation are disseminated through the CNS, primarily in the white matter, with a predilection for the lateral ad posterior columns (especially in the cervical and dorsal regions), the optic nerves, and periventricular areas. Tracts in the midbrain, pons, and cerebellum also are affected, and gray matter in both cerebrum and cord may be affected.

Cell bodies and axons are usually preserved, especially in early lesions. Later, axons may be destroyed, especially in the long tracts, and a fibrous gliosis gives the tracts their “sclerotic” appearance. Both early and late lesions may be found simultaneously. Chemical changes in lipid and protein constituents of myelin have been demonstrated in and around the plaques.

Various symptoms and signs of CNS dysfunction, with remissions and recurring exacerbations, characterize the disease. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized.

The course is highly varied, unpredictable, and, in most patients, remittent. Life span is probably not shortened except in the most severe cases. At first, months or years of remission may separate episodes, especially when the disease begins with retrobulbar optic neuritis. Remissions can last >10 years. However, some patients have frequent attacks and are rapidly incapacitated; for a few, particularly for male patients with onset in middle age, the course can be rapidly progressive. Exposure to excess heat from fever or the environment sometimes worsens symptoms.

Diagnosis is indirect, by deduction from clinical and laboratory features. MRI, the most sensitive diagnostic imaging technique, may show plaques. Gadolinium-contrast enhancement can distinguish areas of active inflammation from older brain plaques. MS lesions may also be visible on contrast-enhanced CT scans, in which sensitivity may be increased by giving twice the iodine dose and delaying scanning (double-dose delayed CT scan).

CSF is abnormal in the majority of patients. IgG may be >13%, and lymphocytes and protein content may be slightly increased. Oligoclonal bands, which indicate IgG synthesis within the blood-brain barrier, may be detected by agarose electrophoresis of CSF in up to 90% of patients with MS, but absence of these bands does not rule out MS. IgG levels correlate with disease severity. Myelin basic protein may be elevated during active demyelination.

Spontaneous remissions and fluctuating symptoms make treatments difficult to evaluate. Corticosteroids are the main form of therapy. They may shorten the symptomatic period during attacks, although they may not affect eventual long-term disability. Patients presenting with acute severe optic neuritis may delay the onset of MS by using high-dose IV corticosteroids.

Immunosuppressive drugs (methotrexate, azathioprine, cyclophosphamide, cladribine) for more severe progressive forms may be used. Immunomodulatory therapy with interferon-β reduces the frequency of relapses in MS. Other promising treatments still under investigation include other interferons, oral myelin, and glatiramer to help keep the body from attacking its own myelin. Glatiramer is a synthetic co-polymer with similarities to myelin basic protein and is administered by daily subcutaneous injection. Its main action is thought to be suppression of the immune is response against myelin to promote immune tolerance (Clegg and Bryant, 2001).

Interferons are cytokines, i.e. soluble proteins that transmit messages between cells and play an essential role in the immune system by helping to destroy microorganisms that cause infection and repairing any resulting damage. Interferons are naturally secreted by infected cells and were first identified in 1957. Their name is derived from the fact that they “interfere” with viral replication and production.

Interferons exhibit both antiviral and antiproliferative activity. On the basis of biochemical and immunological properties, the naturally-occurring human interferons are grouped into three major classes: interferon-alpha (leukocyte), interferon-beta (fibroblast) and interferon-gamma (immune). Alpha-interferon is currently approved in the United States and other countries for the treatment of hairy cell leukemia, venereal warts, Kaposi's Sarcoma (a cancer commonly affecting patients suffering from Acquired Immune Deficiency Syndrome (AIDS)), and chronic non-A, non-B hepatitis.

Further, interferons (IFNs) are glycoproteins produced by the body in response to a viral infection. They inhibit the multiplication of viruses in protected cells. Consisting of a lower molecular weight protein, IFNs are remarkably non-specific in their action, i.e. IFN induced by one virus is effective against a broad range of other viruses. They are however species-specific, i.e. IFN produced by one species will only stimulate antiviral activity in cells of the same or a closely related species. IFNs were the first group of cytokines to be exploited for their potential anti-tumor and antiviral activities.

The three major IFNs are referred to as IFN-α, IFN-β and IFN-γ. Such main kinds of IFNs were initially classified according to their cells of origin (leukocyte, fibroblast or T cell). However, it became clear that several types might be produced by one cell. Hence leukocyte IFN is now called IFN-α, fibroblast IFN is IFN-β and T cell IFN is IFN-γ. There is also a fourth type of IFN, lymphoblastoid IFN, produced in the “Namalwa” cell line (derived from Burkitt's lymphoma), which seems to produce a mixture of both leukocyte and fibroblast IFN.

The interferon unit or international unit for interferon (U or IU, for international unit) has been reported as a measure of IFN activity defined as the amount necessary to protect 50% of the cells against viral damage. The assay that may be used to measure bioactivity is the cytopathic effect inhibition assay as described (Rubinstein, et al. 1981; Familletti, P. C., et al., 1981). In this antiviral assay for interferon about 1 unit/ml of interferon is the quantity necessary to produce a cytopathic effect of 50%. The units are determined with respect to the international reference standard for Hu-IFN-beta provided by the National Institutes of Health (Pestka, S. 1986).

Every class of IFN contains several distinct types. IFN-β and IFN-γ are each the product of a single gene.

The proteins classified as IFNs-α are the most diverse group, containing about 15 types. There is a duster of IFN-α genes on chromosome 9, containing at least 23 members, of which 15 are active and transcribed. Mature IFNs-α are not glycosylated.

IFNs-α and IFN-β are all the same length (165 or 166 amino adds) with similar biological activities. IFNs-γ are 146 amino adds in length, and resemble the α and β classes less closely. Only IFNs-γ can activate macrophages or induce the maturation of killer T cells. These new types of therapeutic agents can are sometimes called biologic response modifiers (BRMs), because they have an effect on the response of the organism to the tumor, affecting recognition via immunomodulation.

Human fibroblast interferon (IFN-β) has antiviral activity and can also stimulate natural killer cells against neoplastic cells. It is a polypeptide of about 20,000 Da induced by viruses and double-stranded RNAs. From the nucleotide sequence of the gene for fibroblast interferon, cloned by recombinant DNA technology, (Derynk et al. 1980) deduced the complete amino acid sequence of the protein. It is 166 amino acid long.

Shepard et al. (1981) described a mutation at base 842 (Cys→Tyr at position 141) that abolished its anti-viral activity, and a variant clone with a deletion of nucleotides 1119-1121.

Mark et al. (1984) inserted an artificial mutation by replacing base 469 (T) with (A) causing an amino acid switch from Cys→Ser at position 17. The resulting IFN-β was reported to be as active as the ‘native’ IFN-β and stable during long-term storage (−70° C.).

Rebif® (recombinant human interferon-β) is the latest development in interferon therapy for multiple sclerosis (MS) and represents a significant advance in treatment. Rebif® is interferon (IFN)-beta 1a, produced from mammalian cell lines. It was established that interferon beta-1a given subcutaneously three times per week is efficacious in the treatment of Relapsing-Remitting Multiple Sclerosis (RR-MS). Interferon beta-1a can have a positive effect on the long-term course of MS by reducing number and severity of relapses and reducing the burden of the disease and disease activity as measured by MRI (Study Group, 1998).

Ribavirin (1-β-D-ribofuranosyl-1H-1,2,4-Triazole-3-carboxamide), the first synthetic broad-spectrum antiviral nucleoside, described in the Merck Index, Eleventh edition as compound no. 8199, is a competitive inhibitor of inosine monophosphate dehydrogenase (IMPDH). It is commercially available e.g. from ICN Pharmaceuticals, Inc., Costa Mesa, Calif. Ribavirin may be prepared as described in U.S. Pat. No. 4,138,547 or U.S. Pat. No. 3,991,078. Its manufacture and formulation are described in U.S. Pat. No. 4,211,771.

The main toxicity associated with Ribavirin administration is a dose-related anemia that is reversible upon cessation of treatment (Di Bisceglie A M et al., 1992; Jarvis, S. et al., 1998). Ribavirin is also known to induce a delay in cellular proliferation (Joksic G. et al., 2000). Many other triazole-type nucleoside analogs used in antiviral and antineoplastic treatments are known to have low level of selectivity for IMPDH, jeopardizing long-term treatments and/or treatments in relatively high dosages.

So far, Ribavirin has been widely used as monotherapy to treat viral infections including respiratory syncytial virus (Hall C B et al., 1985), Lassa fever virus (McCormick J B, et al., 1986) influenza (Togo Y, McCracken E A., 1976) and hepatitis C (Reichard O, et al., 1991; Di Bisceglie A M et al., 1992;). It has also been found that the combination of Ribavirin with interferon-alpha (IFNs-α) was more effective for the treatment of hepatitis C than Ribavirin alone (Brillanti s., et al., 1994). In addition to its well-known role as a direct antiviral agent, Ribavirin also exhibits immunomodulatory properties (Hultgren C. et al., 1998; Tam R C et al., 1999).

EP11329393 and WO01/66034 describe the synthesis and use of Ribavirin analogs (L-isomers, Levovirin) for the treatment of infections e.g. hepatitis B virus, parasitic infestations e.g. protozoan or helminth, neoplasms e.g cancers or tumors caused by a virus or autoimmune diseases e.g. arthritis, psoriasis or multiple sclerosis, alone or in combination with an anti-viral agent (e.g. interferon-alpha, interferon-gamma, Ribavirin, acyclovir), an anti-fungal agent, an ant-tumor agent, a dermatologic agent, a migraine preparation or steroids.

WO00/30656 describes a method for treating neurological diseases such as Alzheimer's disease, Parkinson's disease, multiple sclerosis and huntington's disease using a combination of Ribavirin and neutrophic factors such as NGF (Nerve Gwoth Factor) or FGF (Fibroblast Growth Factor).

The treatment of demyelinating diseases with a combination of Ribavirin and is IFN-β has not yet been considered in the art.

### SUMMARY OF THE INVENTION

The present invention relates to the use of a compound of formula (I) 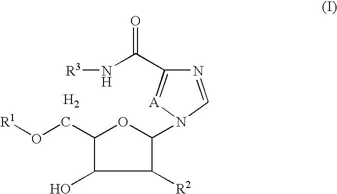
wherein R¹, R², R³ and A are described in details in the description hereinafter, in combination with an interferon (IFN), or an isoform, mutein, fused protein, functional derivative, active fraction or salt thereof, for the manufacture of a medicament for treatment and/or prevention of a demyelinating disease, for simultaneous, sequential or separate use.

### DETAILED DESCRIPTION OF THE INVENTION

The invention relates to the use of compounds of formula (I) 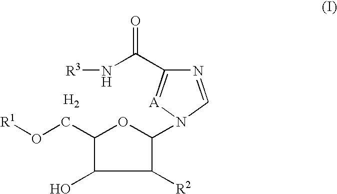
in combination with an interferon (IFN), or an isoform, mutein, fused protein, functional derivative, active fraction or salt thereof, for the manufacture of a medicament for treatment and/or prevention of a demyelinating disease, for simultaneous, sequential or separate use.

The substituents within formula (I) are defined as follows:

R¹ is selected from the group comprising or consisting of hydrogen, acyl, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl aminocarbonyl, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, aryl, heteroaryl, an unsubstituted or substituted C₃-C₈-cycloalkyl or an unsubstituted or substituted C₃-C₈ membered heterocycloalkyl, an unsubstituted or substituted C₁-C₆-alkyl aryl, an unsubstituted or substituted C₁-C₆-alkyl heteroaryl, an unsubstituted or substituted C₁-C₆ alkyl cycloalkyl containing optionally 1-3 heteroatoms, an unsubstituted or substituted C₂-C₆-alkenyl-aryl or -heteroaryl, an unsubstituted or substituted C₂-C₆-alkynyl aryl or -heteroaryl, sulfonyl or phosphoryl.

R² is selected from the group comprising or consisting of hydrogen, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkoxy, hydroxy, halogen.

R³ is selected from the group comprising or consisting of hydrogen, an unsubstituted or substituted C₁-C₆-alkyl, an unsubstituted or substituted C₂-C₆-alkenyl, an unsubstituted or substituted C₂-C₆-alkynyl.

A is N or CR⁴.

R⁴ is H or NR⁵R^{5′}.

R⁵ and R^{5′} are independently from each other selected from the group comprising or consisting of hydrogen, acyl, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl aminocarbonyl, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, aryl, heteroaryl, an unsubstituted or substituted C₃-C₈-cycloalkyl or an unsubstituted or substituted C₃-C₈ membered heterocycloalkyl, an unsubstituted or substituted C₁-C₆-alkyl aryl, an unsubstituted or substituted C₁-C₆-alkyl heteroaryl, an unsubstituted or substituted C₁-C₆ alkyl cycloalkyl containing optionally 1-3 heteroatoms, an unsubstituted or substituted C₂-C₆-alkenyl-aryl or -heteroaryl, an unsubstituted or substituted C₂-C₆-alkynyl aryl or -heteroaryl, sulfonyl or phosphoryl;

R³ and R⁵ may form a heterocyclic ring together.

The following paragraphs provide definitions of the various chemical moieties that make up the compound used in the invention and are intended to apply uniformly through-out the specification and claims unless an otherwise expressly set out definition provides a broader definition.

“Acyl” refers to the group —C(O)R where R includes “C₁-C₆-alkyl”, “aryl”, “heteroaryl”, “C₁-C₆-alkyl aryl” or “C₁-C₆-alkyl heteroaryl”.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C═CH), propargyl (—CH₂C≡CH), and the like.

“Aminocarbonyl” refers to the group —C(O)NRR′ where each R, R′ includes independently hydrogen or C₁-C₆-alkyl or aryl or heteroaryl or “C₁-C₆-alkyl aryl” or “C₁-C₆-alkyl hetero-aryl”.

“C₁-C₆-alkyl aminocarbonyl” refers to C₁-C₆-alkyl groups having an aminocarbonyl substituent, including 2-(dimethylaminocarbonyl)ethyl and the like.

“Amino” refers to the group —NRR′ where each R, R′ is independently hydrogen or “C₁-C₆-alkyl” or “aryl” or “heteroaryl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl amino” refers to C₁-C₅-alkyl groups having an amino substituent, including 2-(1-pyrrolidinyl)ethyl and the like.

“C₁-C₆-alkyl alkoxy” refers to C₁-C₆-alkyl groups having an alkoxy substituent, including 2-ethoxyethyl and the like.

“C₁-C₆-alkyl sulfanyl” refers to C₁-C₅-alkyl groups having a sulfanyl substituent, including 2-(ethylsulfanyl)ethyl and the like.

“C₁-C₆-alkyl sulfinyl” refers to C₁-C₅-alkyl groups having a sulfinyl substituent, including 2-(methylsulfinyl)ethyl and the like.

“C₁-C₈-alkyl sulfonyl” refers to C₁-C₅-alkyl groups having a sulfonyl substituent, including 2-(methylsulfonyl)ethyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Preferred heterocycloalkyl include pyrrolidine, piperidine, piperazine, 1-methylpiperazine, morpholine, and the like.

“C₁-C₆-alkyl aryl” refers to C₁-C₆-alkyl groups having an aryl substituent, including benzyl, phenethyl and the like.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₁-C₆-alkyl cycloalkyl” refers to C₁-C₆-alkyl groups having a cycloalkyl substituent including cyclohexylmethyl, cyclopentylpropyl, and the like.

“C₂-C₆-alkenyl aryl” refers to C₂-C₈-alkenyl groups having an aryl substituent, including 2-phenylvinyl and the like.

“C₂-C₈-alkenyl heteroaryl” refers to C₂-C₆-alkenyl groups having a heteroaryl substituent, including 2-(3-pyridinyl)vinyl and the like.

“C₂-C₆-alkynyl aryl” refers to C₂-C₈-alkynyl groups having an aryl substituent, including phenylethynyl and the like.

“C₂-C₆-alkynyl heteroaryl” refers to C₂-C₈-alkynyl groups having a heteroaryl substituent, including 2-thienylethynyl and the like.

“Sulfanyl” refers to groups —S—R where R includes H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Sulfinyl” refers to a group “—S(O)—R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Phosphoryl” refers to the group —PORR′ where each R, R′ is independently hydrogen, OH, alkoxy, alkyl.

“Alkoxy” refers to the group OR where R includes “C₁-C₆-alkyl” or “aryl” or “hetero-aryl” or “C₁-C₆-alkyl aryl” or “C₁-C₆-alkyl heteroaryl”. Preferred alkoxy groups include by way of example, methoxy, ethoxy, phenoxy and the like.

“Halogen” refers to fluoro, chloro, bromo and iodo atoms.

“Substituted or unsubstituted”: Unless otherwise constrained by the definition of the indi-vidual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

In the following, the compounds of formula (I) may also be referred to as the “compound(s) of the invention”.

The term “prevention” within the context of this invention refers not only to a complete prevention of the disease or one or more symptoms of the disease, but also to any partial or substantial prevention, attenuation, reduction, decrease or diminishing of the effect before or at early onset of disease.

The term “treatment” within the context of this invention refers to any beneficial effect on progression of disease, including attenuation, reduction, decrease or diminishing of the pathological development after onset of disease.

A “demyelinating disease”, as used in the context of the present invention, is a disease involving abnormalities in myelin sheaths of the nervous system, in particular destruction of myelin, as described in detail in the “Background of the Invention” above.

An “interferon” or “IFN”, as used herein, is intended to include any molecule defined as such in the literature, comprising for example any types of IFNs mentioned in the above section “Background of the Invention”. In particular, IFN-α, IFN-β and IFN-γ are included in the above definition. IFN-β is the preferred IFN according to the present invention. IFN-β suitable in accordance with the present invention is commercially available e.g. as Rebif® (Serono), Avonex® (Biogen) or Betaferon® (Schering). The use of interferons of human origin is also preferred in accordance with the present invention. The term interferon, as used herein, is intended to encompass salts, functional derivatives, variants, analogs and active fragments thereof.

The term “interferon-beta (IFN-β)”, as used herein, is intended to include fibroblast interferon in particular of human origin, as obtained by isolation from biological fluids or as obtained by DNA recombinant techniques from prokaryotic or eukaryotic host cells, as well as its salts, functional derivatives, variants, analogs and active fragments.

As used herein the term “muteins” refers to analogs of IFN in which one or more of the amino acid residues of a natural IFN are replaced by different amino acid residues, or are deleted, or one or more amino acid residues are added to the natural sequence of IFN, without changing considerably the activity of the resulting products as compared to the wild type IFN. These muteins are prepared by known synthesis and/or by site-directed mutagenesis techniques, or any other known technique suitable therefore. Preferred muteins include e.g. the ones described by Shepard et al. (1981) or Mark et al. (1984).

Any such mutein preferably has a sequence of amino acids sufficiently duplicative of that of IFN, such as to have substantially similar or even better activity to an IFN. The biological function of interferon is well known to the person skilled in the art, and biological standards are established and available e.g. from the National Institute for Biological Standards and Control (http://immunology.org/links/NIBSC).

Bioassays for the determination of IFN activity have been described. An IFN assay may for example be carried out as described by Rubinstein et al., 1981. Thus, it can be determined whether any given mutein has substantially a similar, or even a better, activity than IFN by means of routine experimentation.

Muteins of IFN, which can be used in accordance with the present invention, or nucleic acid coding therefore, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino add substitutions of polypeptides or proteins of the invention, may include synonymous amino adds within a group, which have sufficiently similar physicochemical properties that substitution between members of the group will preserve the biological function of the molecule. It is clear that insertions and deletions of amino acids may also be made in the above-defined sequences without altering their function, particularly if the insertions or deletions only involve a few amino adds, e.g., under thirty, and preferably under ten, and do not remove or displace amino adds which are critical to a functional conformation, e.g., cysteine residues. Proteins and muteins produced by such deletions and/or insertions come within the purview of the present invention.

Preferably, the synonymous amino add groups are those defined in Table I. More preferably, the synonymous amino acid groups are those defined in Table II; and most preferably the synonymous amino add groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of IFN, for use in the present invention include any known method steps, such as presented in U.S. Pat. Nos. 4,959,314, 4,588,585 and 4,737,462, to Mark et al; U.S. Pat. No. 5,116,943 to Koths et al., U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al). Specific muteins of IFN-beta have been described, for example by Mark et al., 1984.

The term “fused protein” refers to a polypeptide comprising an IFN, or a mutein thereof, fused to another protein, which e.g., has an extended residence time in body fluids. An IFN may thus be fused to another protein, polypeptide or the like, e.g., an immunoglobulin or a fragment thereof.

“Functional derivatives” as used herein cover derivatives of IFN, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity IFN, and do not confer toxic properties on compositions containing it. These derivatives may, for example, include polyethylene glycol side-chains, which may mask antigenic sites and extend the residence of IFN in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of IFN, or muteins and fused proteins, the present invention covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has no significantly reduced activity as compared to the corresponding IFN.

The term “salts” herein refers to both salts of carboxyl groups and to acid addition salts of amino groups of the proteins described above or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of the proteins (IFN) relevant to the present invention, i.e., the ability to bind to the corresponding receptor and initiate receptor signaling.

Demyelinating diseases according to the invention may be e.g. multiple sclerosis, acute disseminated encephalomyelitis, acute inflammatory peripheral neuropathies adrenoleukodystrophy and adrenomyeloneuropathy, Leber's hereditary optic atrophy, or HTLV-associated myelopathy, as described in the introduction. They may preferably be neuropathies with abnormal myelination. They may concern the peripheral or the central nervous system.

The most common demyelinating disease is multiple sclerosis. Therefore, in a preferred embodiment of the invention, the combination of the compounds of the invention and an interferon is used for treatment and/or prevention of multiple sclerosis (MS). In accordance with the present invention, MS may have a chronic progressive disease development. It may also be relapsing-remitting multiple sclerosis or any intermediate manifestation of the disease.

According to one embodiment of the invention, R¹ of formula (I) is H. In another embodiment R² is OH. In a further embodiment A is N. In yet a further embodiment of the invention R³ and R⁵ form a 6-membered heterocyclic ring. In a preferred embodiment of the invention, the heterocyclic ring is a pyrimidine or a pyrimidine-one.

In accordance with the present invention, the use of Ribavirin (1-β-D-ribofuranosyl-1H-1,2,4-Triazole-3-carboxamide), as compound of the invention is especially preferred.

In accordance with the present invention, the use of recombinant human IFN-beta and the compounds of the invention is further particularly preferred.

A special kind of interferon variant has been described recently. The so-called “consensus interferons” are non-naturally occurring variants of IFN (U.S. Pat. No. 6,013,253). Consensus interferons were shown to be effective in the treatment of multiple sclerosis.

Therefore, in a preferred embodiment of the invention, the compounds of the invention are used in combination with a consensus interferon.

As used herein, human interferon consensus (IFN-con) shall mean a non-naturally-occurring polypeptide, which predominantly includes those amino acid residues that are common to a subset of IFN-alpha's representative of the majority of the naturally-occurring human leukocyte interferon subtype sequences and which includes, at one or more of those positions where there is no amino acid common to all subtypes, an amino acid which predominantly occurs at that position and in no event includes any amino acid residue which is not existent in that position in at least one naturally-occurring subtype. IFN-con encompasses but is not limited to the amino acid sequences designated IFN-con1, IFN-con2 and IFN-con3 which are disclosed in U.S. Pat. Nos. 4,695,623, 4,897,471 and 5,541,293. DNA sequences encoding IFN-con may be produced as described in the above-mentioned patents, or by other standard methods.

In a further preferred embodiment, the fused protein comprises an Ig fusion. The fusion may be direct, or via a short linker peptide which can be as short as 1 to 3 amino add residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of IFN and the immunoglobulin sequence. The resulting fusion protein may have improved properties, such as an extended residence time in body fluids (half-life), increased specific activity, increased expression level, or the purification of the fusion protein is facilitated.

In a further preferred embodiment, IFN is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂, IgG₃ or IgG₄, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

Standard dosages of human IFN-beta presently used in the treatment of relapsing-remitting MS are ranging from 80 000 IU/kg and 200 000 IU/kg per day or 6 MIU (million international units) and 12 MIU per person per day or 22 to 44 μg (microgram) per person. In accordance with the present invention, IFN may preferably be administered at a dosage of about 1 to 50 μg, more preferably of about 10 to 30 μg or about 10 to 20 μg per person per day. The preferred route of administration is is subcutaneous administration, administered e.g. three times a week. A further preferred route of administration is the intramuscular administration, which may e.g. be applied once a week.

Preferably 22 to 44 μg or 6 MIU to 12 MIU of IFN-beta is administered three times a week by subcutaneous injection.

IFN-beta may be administered subcutaneously, at a dosage of 250 to 300 μg or 8 MIU to 9.6 MIU, every other day.

30 μg or 6 MIU IFN-beta may further be administered intramuscularly once a week.

IFN-beta may also be administered daily or every other day, of less frequent. Preferably, IFN-beta is administered one, twice or three times per week

The administration of active ingredients in accordance with the present invention may be by intravenous, intramuscular or subcutaneous route. The preferred route of administration for IFN-beta is the subcutaneous route.

In a preferred embodiment the compound of the invention, preferably Ribavirin, is administered at a dosage of about 100 to 20 00 mg per person per day, preferably of about 400 to 1200 mg per person per day, more preferably about 800 to 1000 mg per person per day, or about 1000 to 1200 mg per person per day. For patients weighing less than 65 kg the usual dose is 800 mg per day, for patients weighing 65 to 85 kg the usual dose is 1000 mg per day and for patients weighning more than 85 kg the usual dose is 1200 mg per day. The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

In a preferred embodiment, the compound of the invention, preferably Ribavirin is administered orally.

Ribavirin may be administered by injection or, preferably, orally. Depending on the mode of administration, the compound can be formulated with the appropriate diluents and carriers to form ointments, creams, foams, and solutions having from about 0.01% to about 15% by weight preferably from about 1% to about 10% by weight of the compound. For injection, Ribavirin is in the form of a solution or suspension, dissolved or suspended in physiologically compatible solution from about 10 mg/ml to about 1500 mg/ml. Injection may be intravenous, intermuscular, intracerebral, subcutaneous, or intraperitoneal.

For oral administration, Ribavirin may be in capsule, tablet, oral suspension, or syrup form. The tablet or capsules may contain from about 10 to 500 mg of Ribavirin. Preferably they may contain about 300 mg of Ribavirin. The capsules may be the usual gelatin capsules and may contain, in addition to the Ribavirin in the quantity indicated above, a small quantity, for example less than 5% by weight, magnesium stearate or other excipient. Tablets may contain the foregoing amount of the compound and a binder, which may be a gelatin solution, a starch paste in water, polyvinyl pyrilidone, polyvinyl alcohol in water, etc. with a typical sugar coating.

Corticosteroids are therapeutically efficacious in the treatment of demyelinating diseases. Therefore, the medicament of the invention may further comprise a corticosteroid, for simultaneous, sequential, or separate use. As corticosteroid treatment, oral prednisone 60 to 100 mg/day tapered over 2 to 3 weeks or IV methylprednisolone 500 to 1000 mg/day for 3 to 5 days may be administered, for instance.

Glatiramer is a synthetic co-polymer with similarities to myelin basic protein and is administered by daily subcutaneous injection. It has also been proved to have a therapeutic effect in multiple sclerosis. In a preferred embodiment of the invention, the medicament further comprises glatiramer, for sequential, separate or simultaneous use.

The compounds of the invention and IFN may be formulated in a pharmaceutical composition.

The term “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The subcutaneous route is preferred in accordance with the present invention.

Another possibility of carrying out the present invention is to activate endogenously the genes for IFN. In this case, a vector for inducing and/or enhancing the endogenous production of IFN in a cell normally silent for express ion of IFN, or which expresses amounts of IFN which are not sufficient, are is used for treatment of a demyelinating disease. The vector may comprise regulatory sequences functional in the cells desired to express IFN. Such regulatory sequences may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “endogenous gene activation” (EGA), and it is described e.g. In WO 91/09955.

The invention further relates to the use of a cell that has been genetically modified to produce IFN in the manufacture of a medicament for the treatment and/or prevention of neurological diseases.

For parenteral (e.g. intravenous, subcutaneous, intramuscular) administration, IFN can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the IFN according to the invention can also be ameliorated by using conjugation procedures which increase the half-life of the molecule in the human body, for example linking the molecule to polyethylenglycol, as described in the PCT Patent Application WO 92/13095.

The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the compounds of the invention and IFN can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning of a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art

Having now described the invention, it will be more readily understood by reference to the following examples that are provided by way of illustration and are not intended to be limiting of the present invention.

### EXAMPLE

Effect of Ribavirin Alone, or in Combination with IFN-Beta, in an in Vivo Model of Multiple Sclerosis

The effect of Ribavirin, either alone or in combination with IFN-beta, on disease development is assayed using an established animal model of multiple sclerosis (MS). The experimental autoimmune encephalomyelitis (EAE) model is a murine chronic demyelinating model. The used method for the induction of EAE in mouse is adapted from the protocol published by Sahrbacher et al. (1998).

EAE Induction Protocol

Mice

Species, strain, substrain and sex: C57 BL/6JICO female mice from IFFA CREDO (Saint Germain sur l'Arbresle, France) colony supplied by Charles River Italia (Calco, Lecco, Italy).

Age and body weight (at randomization): About 8-week old; 18-22 g.

Housing: animals are kept in the following conditions:
10 animals/cage in air-conditioned rooms
Temperature: 22° C.±2
Relative humidity: 55%±10
Air changes: about 15-20/hour filtered on HEPA 99.99%.
Light: 12-hour cycle (7 a.m.-7 p.m.)
Cage: Makrolon® cage 42.5×26.6×15 each fitted with a stainless steel cover-feed rack. A grill is inserted on the cage bottom. The waste that drops through the grill onto the cage bottom is periodically disposed.

Diet: GLP 4RF25 top certificate pelleted diet produced by Charles River Italia's feed licensee Mucedola S.r.l., Settimo Milanese. To facilitate nourishment of sick animals, from day 7 wet pellets are placed every day on the cage bottom. The Producer supplies a certificate of analysis for nutrients and contaminants, the levels of which are within the limits proposed by EPA-TSCA (44FR:44053-44093. Jul. 26, 1979). The diet is available “ad libitum” to the animals.

Water From the municipal main watering system. Water is filtered and distributed “ad libitum” to the animals by an automatic valve system. Plastic bottles are used in addition to the automatic watering system. Periodically drinking water is analyzed for microbiologic count heavy metals, other contaminants (e.g. solvents, pesticides) and other chemical and physical characteristics. The acceptance limits of quality of the drinking water are those defined in the EEC Directive 80/778.

Immunization Procedure

Experimental autoimmune encephalomyelitis (EAE) is induced in groups of mice as follows: 6 groups of 10 female mice are immunized (day=0) by injecting subcutaneously (s.c.) in the left flank 0.2 mL of an emulsion composed of 200 μg MOG₃₅₋₅₅ peptide (Neosystem, Strasbourg, France) in Complete Freund's Adjuvant (CFA, Difco, Detroit, U.S.A.) containing 0.5 mg of Mycobacterium tuberculosis.

Immediately after, they receive an intraperitoneal (i.p.) injection of 500 ng pertussis toxin (List Biological Lab., Campbell, Calif., U.S.A.) dissolved in 400 μL of buffer (0.5 M NaCl, 0.017% Triton X-100, 0.015 M Tris, pH=7.5).

On day 2, the animals are given a second i.p. Injection of 500 ng pertussis toxin.

On day 7, the mice receive a second dose of 200 μg of MOG₃₅₋₅₅ peptide in CFA injected s.c. in the right flank. Starting approximately from day 8-10 this procedure results in a gradually progressing paralysis, arising from the tail and ascending up to the forelimbs.

The treatment is started for each animal at the appearance of a clinical score≧1 and is subsequently continued for 60 consecutive days. The animals are treated dally as follows:

| | No. of | Test | | | Administration | Administration |
|---|---|---|---|---|---|---|
| Group | Mice | Substance | Dose | | route | volume/rate |
| 1 | 10 | r-IFN-β | 20,000 | U/mouse | i.p. or s.c. | 200 μL/mouse |
| 2 | 10 | Ribavirin | 50 | mg/kg | i.p. or s.c. | 100 μL |
| 3 | 10 | Ribavirin | 100 | mg/kg | i.p. or s.c. | 200 μL |
| 4 | 10 | r-IFN-β + | 20,000 | U/mouse + | i.p. or s.c. + | 200 μL/mouse |
| | | Ribavirin | 50 | mg/kg | i.p. or s.c. | 100 μL |
| 5 | 10 | r-IFN-β + | 20,000 | U/mouse + | i.p. or s.c. + | 200 μL/mouse |
| | | Ribavirin | 100 | mg/kg | i.p. or s.c. | 200 μL |

| | | | | | |
|---|---|---|---|---|---|
| 6 | 10 | PBS | PBS | i.p. or s.c. | 200 μL |
| | (controls) | | | | |

PBS is used as vehicle to dilute Ribavirin and r-mIFN-β to the appropriate concentration.

mIFN-β is administered daily by s.c. or i.p. route at the dosage of 20,000 U/mouse in a volume of 200 μL/mouse daily. Ribavirin is administered daily i.p. or i.c. at two different dosages, 50 mg/kg or 100 mg/kg, alone or in combination with mIFN-β by s.p. or i.p. route at the dosage of 20,000 U/mouse, in a volume of 200 μL/mouse.

Clinical signs and body weight of the animals are monitored daily in each group of treatment. Starting from day 7, the animals are individually examined for the presence of paralysis by means of the following clinical score:
0=no sign of disease
0.5=partial tail paralysis
1=tail paralysis
1.5=tail paralysis+partial unilateral hindlimb paralysis
2=tail paralysis+hindlimb weakness or partial hindlimb paralysis
2.5=tail paralysis+partial hindlimb paralysis (lowered pelvi)
3=tail paralysis+complete hindlimb paralysis
3.5=tall paralysis+complete hindlimb paralysis+incontinence
4=tail paralysis+hindlimb paralysis+weakness or partial paralysis of forelimbs
5=moribund or dead

Histological Analysis

At the end of treatment, the animals, under pentobarbital anesthesia, are perfusion-fixed with 4% formaldehyde via the left ventricle. Subsequently, their spinal cords is carefully dissected out and fixed in formalin. Spinal cord slices are embedded in paraffin blocks. Sectioning and staining with hematoxylin and eosin for inflammation, and with Kluver-PAS (Luxol fast blue plus Periodic Acid Schiff staining) for the detection of demyelination, is performed.

Results of clinical examinations is expressed as the mean(±SEM) score within each group. The effects of the test substances are compared with the respective vehicle-treated positive control group. Differences of clinical score values among groups is analysed by One-way ANOVA test followed in case of significance by the Fisher test at each measurement time. Body weight data will are evaluated by one-way ANOVA and in case of significance followed by the Tukey test. The S-Plus® software is used.

It is anticipated that a significant effect of the combined treatment with IFN-β and Ribavirin can be observed.

### REFERENCES

1. Study Group. The Lancet 1998; 352, 1498-1504.
2. Brillanti S, Garson J, Foli M, Whitby K, Deaville R, Masci C, Migiloli M, Barbara. A pilot study of combination therapy with ribavirin plus interferon alfa for interferon alfa-resistant chronic hepatitis C. L. Gastroenterology 1994 September; 107(3): 812-7.
3. Clegg end Bryant, Exp. Opin. Parmacother 2000; 2(4): 623-639.
4. Derynk R. et al., Nature 1980; 285, 542-547.
5. Di Bisceglie A M, Shindo M, Fong T L, Fried M W, Swain M G, Bergasa N V, Axiotis C A, Waggoner J G, Park Y, Hoofnagle J H. A pilot study of ribavirin therapy for chronic hepatitis C. Hepatology 1992; 16(3):649-54.
6. Familletti, P. C., Rubinstein, S., and Pestka, S. 1981 “A Convenient and Rapid Cytopathic Effect Inhibition Assay for Interferon,” in Methods in Enzymology, Vol. 78 (S. Pestka, ed.), Academic Press, New York, 387-394;
7. Hall C B, McBride J T, Gala C L, Hildreth S W, Schnabel K C. Ribavirin treatment of respiratory syncytial viral infection in infants with underlying cardiopulmonary disease. JAMA 1985 Dec. 6; 254(21):3047-51.
8. Hultgren C, Milich D R, Welland O, Sallberg M. (1998). The antiviral compound ribavirin modulates the T helper (Th) 1/Th2 subset balance in hepatitis B and C virus-specific immune responses. J Gen Virol 1998; 79:2381-2391.
9. Jarvis S M, Thom J A, Glue P. Ribavirin uptake by human erythrocytes and the involvement of nitrobenzylthioinosine-sensitive (es)-nucleoside transporters. Br J Pharmacol. 1998; 123(8):1587-92.
10. Joksic G, Stankovic M, Vasic V, Cakar M, Jokanovic M. Influence of ribavirin on the micronucleus formation and in vitro proliferation of human lymphocytes. Neoplasma. 2000; 47(5):283-7.
11. McCormick J B, King I J, Webb P A, Scribner C L, Craven R B, Johnson K M, Elliott L H, Belmont-Williams R. Lassa fever. Effective therapy with ribavirin. N Engl J Med. 1986 Jan. 2; 314(1):20-6.
12. Mark D. F. et al., Proc. Natl. Acad. Sci. U.S.A., 81 (18) 5662-5666 (1984).
13. Pestka, S. (1986) “Interferon Standards and General Abbreviations, in Methods in Enzymology (S. Pestka, ed.), Academic Press, New York 119, 14-23.
14. Reichard O, Andersson J, Schvarcz R, Weiland O. Ribavirin treatment for chronic hepatitis C. Lancet 1991; 337(8749):1058-61.
15. Reichard O, Schvarcz R, Welland O. Therapy of hepatitis C: alpha interferon and ribavirin. Hepatology. 1997; 26(3 Suppl 1):108S-111S. Review.
16. Rubinstein, S., Familletti, P. C., and Pestka, S. Convenient Assay for Interferons. J. Virol 1981; 37, 755-758.
17. Sahrbacher U C, Lechner F, Eugster H P, Frei K, Lassmann H, Fontana A. Mice with an inactivation of the inducible nitric oxide synthase gene are susceptible to experimental autoimmune encephalomyelitis. Eur J Immunol. 1998 April; 28(4):1332-8.
18. Shepard H. M. et al., Nature 1981; 294, 563-565.
19. Tam R C, Pal B, Bard J, Lim C, Averett D R, Phan U T, Milovanovic T. Ribavirin polarizes human T cell responses towards a Type 1 cytokine profile. J Hepatol. 1999; 30(3):376-82.
20. Togo Y, McCracken E A., 1976. Double-blind clinical assessment of ribavirin (virazole) in the prevention of induced infection with type B influenza virus. J Infect Dis 1976 June; 133 Suppl: A109-13.

1-19. (canceled)

20. A method of treating or preventing a demyelinating disease comprising the administration of a composition comprising a compound of formula (I) 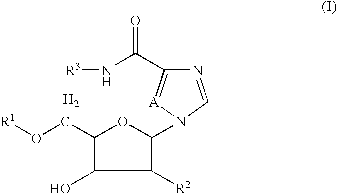
wherein
R¹ is selected from the group comprising or consisting of hydrogen, acyl, C₁-C₆-alkyl, C₂-C₆-alkenyl, C₂-C₆-alkynyl, C₁-C₆-alkyl aminocarbonyl, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, aryl, heteroaryl, C₃-C₈-cycloalkyl or C₃-C₈ membered heterocycloalkyl, C₁-C₆-alkyl aryl, C₁-C₆-alkyl heteroaryl, C₁-C₆ alkyl cycloalkyl containing optionally 1-3 heteroatoms, C₂-C₆-alkenyl-aryl or -heteroaryl, C₂-C₆-alkynyl aryl or -heteroaryl, sulfonyl or phosphoryl;
R² is selected from the group comprising or consisting of hydrogen C₁-C₆-alkyl, C₂-C₆-alkenyl, C₂-C₆-alkynyl, C₁-C₆-alkoxy, hydroxy, halogen;
R³ is selected from the group comprising or consisting of hydrogen, C₁-C₆-alkyl, C₂-C₆-alkenyl, C₂-C₆-alkynyl;
A is N or CR⁴ wherein
R⁴ is H or NR⁵R^{5′} wherein
R⁵ and R^{5′} are independently from each other selected from the group comprising or consisting of hydrogen, acyl, C₁-C₆-alkyl, C₂-C₆-alkenyl, C₂-C₆-alkynyl, C₁-C₆-alkyl aminocarbonyl, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, heteroaryl, C₃-C₈ membered cycloalkyl or heterocycloalkyl, C₁-C₆alkyl aryl, C₁-C₆-alkyl heteroaryl, C₁-C₆ alkyl cycloalkyl containing optionally 1-3 heteroatoms, C₂-C₆-alkenyl-aryl or -heteroaryl, C₂-C₆-alkynyl aryl or -heteroaryl, sulfonyl or phosphoryl.
R³ and R⁵ may form a heterocyclic ring together;
and a composition comprising an interferon-beta (IFN-β), or an isoform, mutein, fused protein, functional derivative, active fraction or salt thereof, to an individual in need of said treatment.

21. The method according to claim 20, wherein R¹ is H.

22. The method according to claim 20, wherein R² is OH.

23. The method according to claim 20, wherein R³ is H.

24. The method according to claim 20, wherein A is N.

25. The method according to claim 20, wherein R³ and R⁵ form a 6-membered heterocyclic ring.

26. The method according to claim 20, wherein the heterocyclic ring is a pyrimidine or a pyrimidine-one.

27. The method according to claim 20, wherein the compound is 1-β-D-ribofuranosyl-1H-1,2,4-triazole-3-carboxamide (Ribavirin).

28. The method according to claim 20, wherein said demyelinating disease is multiple sclerosis.

29. The method according to claim 20, wherein said fused protein comprises an Ig fusion.

30. The method according to claim 20, wherein said functional derivative comprises at least one moiety attached to one or more functional groups, which occur as one or more side chains on the amino acid residues.

31. The method according to claim 30, wherein said moiety is a polyethylene moiety.

32. The method according to claim 20, wherein said IFN-β is administered at a dosage of about 1 to 50 μg per person per day, or about 10 to 30 μg per person per day or about 10 to 20 μg per person per day.

33. The method according to claim 20, wherein said IFN-β is administered daily or every other day.

34. The method according to claim 20, wherein said IFN-β is administered twice or three times per week.

35. The method according to claim 20, wherein said IFN-β is administered subcutaneously.

36. The method according to claim 20, wherein said IFN-β is administered intramuscularly.

37. The method according to claim 20, wherein said compound is administered at a dosage of about 100 to 2000 mg per person per day, or about 400 to 1200 mg per person per day, or about 800 to 1000 mg per person per day, or about 1000 to 1200 mg per person per day.

38. The method according to claim 20, wherein said compound is administered orally.

39. The method according to claim 20, wherein said compositions are administered simultaneously, sequentially, or separately.

40. The method according to claim 20, wherein said compound and said IFN-β are administered as a single composition.

41. The method according to claim 20, wherein said compound and said IFN-β are administered as different compositions.
